(19) United States
(12) Patent Application Publication
(10) Pub. No.: US 2009/0306472 A1
(43) Pub. Date: Dec. 10, 2009

(54) SYSTEMS AND TECHNIQUES FOR ENDOSCOPIC DILATION

(76) Inventors: Charles J. Filipi, Omaha, NE (US); Timothy B. Hunt, Miami Beach, FL (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 12/506,149

(22) Filed: Jul. 20, 2009

Related U.S. Application Data
(63) Continuation-in-part of application No. PCT/US2008/051464, filed on Jan. 18, 2008.
(60) Provisional application No. 60/885,623, filed on Jan. 18, 2007.
(60) Provisional application No. 60/970,819, filed on Sep. 7, 2007.
(60) Provisional application No. 61/221,547, filed on Jun. 29, 2009.

(51) Int. Cl. A61B 1/00; A61M 29/02

(52) U.S. Cl. 600/104; 606/192

(57) ABSTRACT

Dilators configured to be used over the top of conventional endoscopes are disclosed. Dilator 100 includes a dilating surface 340 and an endoscope outlet 342 at its distal end, the inner diameter of which is chosen to closely approximate the outer diameter of the endoscope. In use, the endoscope may serve as a guide to the dilator 100 both during initial and during dilation of, for example, an esophageal stricture. A safety handle 310 is also provided which gives feedback to the operator based on the amount of applied axial force, which helps to reduce the chances of injury.

### RELATED APPLICATION DATA

This application is a continuation in part of PCT/US2008/051464 filed Jan. 18, 2008, which claims the benefit of U.S. Ser. No. 60/885,623 filed Jan. 18, 2007 and U.S. Ser. No. 60/970,819 filed Sep. 7, 2007, the disclosures of which are incorporated by reference. This application also claims the benefit of U.S. Ser. No. 61/221,547 filed Jun. 29, 2009, the disclosure of which is incorporated by reference.

### TECHNICAL FIELD

The present invention is generally related to surgical apparatus and techniques. More particularly, but not exclusively, it is related to novel dilators, overtubes, and tamponade devices for use in endoscopic procedures and novel safety handles that can be used in operating such devices. In one particular form, the invention provides systems and techniques relating to “over the endosope” dilation of constricted body lumens. In another particular form, the invention provides systems and techniques for avoiding the application of excessive axial force during a dilation procedure.

### BACKGROUND

Constriction of natural body lumens, such as those of the human gastrointestinal tract, can occur in numerous ways. Some strictures are caused by muscular spasm, others by disease, and others by injury. Regardless of the cause, the typical method of treatment is to physically dilate the region using a medical device designed for that purpose.

Several types of devices are used for dilation. One generally established type is a bougie. Bougie tubes may be in the form of a mercury- or tungsten-filled tube with a tapered end that gradually opens the strictured esophagus as it is pushed past the treatment site. These devices come in a series of increasing sizes, each tube having a single effective dilating diameter, generally between 10 and 60 French. (French is a measure of circumference based on the diameter in millimeters, mm.) The bougie is typically introduced blindly after the physician has judged the proper beginning size with an endoscope. Some physicians follow a rule of thumb not to dilate a stricture more than three successive French sizes (3 mm) in a single session. If the lumen has not satisfactorily been opened after three sizes, the patient returns at a later time for another treatment session.

Another type of dilating device is a balloon. Balloon dilators may be comprised of polyethylene, and may be introduced through the working channel of an endoscope. The physician views the proximal end of a stricture site with an endoscope and introduces the deflated balloon into the narrowed area. The balloon is then inflated with saline or other fluid to effectively open the stricture site pneumatically. Balloons provide the advantages of multiple dilator diameters with a single intubation, passage through the working channel of an endoscope, and visualization of a stricture site from the proximal end.

Another type of device is a wire-guided dilator. These devices are passed into the patient over a guidewire that has been pre-fed along a lumen of the gastrointestinal tract. The guidewire keeps the tip of the device in the lumen while it is being passed, to avoid perforating through the wall of the lumen.

US 2004/0122462 to Bakos describes a wire-guided dilator that has an endoscope lumen with a transparent section near its distal end. The transparent section allows the endoscope, which is typically inserted after the dilator is in place, to be used to visualize the constriction through the walls of the dilator.

In one aspect, the novel dilators described herein can be used in place of the Bakos dilator for the applications describes therein. However, unlike the wire-guided Bakos dilator, several of the dilator configurations described herein do not require pre insertion of a guidewire. Rather, they are constructed such that they can be inserted directly over an in-place endoscope.

In another aspect, a novel handle is provided for dilatation devices that achieve dilation when a ramped surface is forced through a stricture, for example bougies, over the guidewire dilators, and any of the over the endoscope dilator configurations describe herein. The handle is designed to give feedback to the operator so as to reduce the chance that the operator will apply excessive axial force during a dilation procedure.

### SUMMARY

In one form, the present invention provides novel systems and techniques wherein a dilator can be inserted directly over the endoscope, for example while the endoscope is positioned at or through the site of a gastrointestinal stricture. To enable over the endoscope dilation, the dilator has an endoscope channel that is open to the distal end of the dilator. In use, the dilator is configured such that it may be initially positioned over a section of the endoscope proximal to the endoscope's distal end. The endoscope is then placed into a desired position in a patient, and then the dilator is slid distally along the length of the endoscope towards the distal working end of the endoscope. In this manner, the endoscope may act as a guide during insertion of the dilator. In preferred implementations, the endoscope is positioned through the stricture during dilation and serves to guide the distal end of the dilator through the stricture.

According to another aspect, a dilator can be provided with a force indicating mechanism on its handle to provide feedback to the operator to prevent application of excessive axial force to the dilator. The force indicating mechanism can provide visual, tactical, and/or audible feedback to the operator based on the amount of applied force. In one form, the force indicating mechanism can be a handle that breaks away upon the application of a predefined force. In another form, the handle can be configured to selectively reveal visual indications of applied force. In still another form, the handle can be configured to signal an alarm based on the amount of applied force.

According to another aspect, a dilator can be constructed by attaching several individual dilator segments onto an endoscope and then securing the segments together such that they form a dilator body that can be slid down the endoscope as an integral unit.

According to another aspect, the dilator can include a scale that is slideably positionable along the length of the dilator. The scale has markings that correspond to the relative dilation diameters provided by the dilation portion of the dilator (e.g. the distal end).

These and other aspects are discussed below.

### BRIEF DESCRIPTION OF THE FIGURES

Although the characteristic features of this invention will be particularly pointed out in the claims, the invention itself, and the manner in which it may be made and used, may be better understood by referring to the following description taken in connection with the accompanying figures forming a part thereof.

FIG. 1 is the distal end of a dilator showing an endoscope extending therefrom.

FIG. 2 is a side view of the FIG. 1 dilator.

FIG. 3 is a side view of the FIG. 1 dilator illustrating the placement of markings.

FIGS. 4A and 4B are schematic end views of a side opening dilator being attached over an endoscope.

FIG. 5 is a side view of the FIG. 4B endoscope illustrating the placement of locking tabs along the length to close the side opening.

FIG. 6 is a side view showing a profile of the distal portion of a dilator having diameter reduction in both a proximal and distal direction.

FIG. 7 is a schematic view of a breakaway handle assembly for a dilator.

FIG. 8 is an a cross sectional view of an endoscope lumen that is partially open along the side of a dilator.

FIGS. 9 and 10 are end schematic views showing different orientations of a cut line that can be used to open the endoscope lumen of along the length of a dilator body.

FIGS. 11 and 12 are perspective and exploded illustrations of an over the endoscope dilator configured for treating esophageal strictures.

FIG. 13 is a view of the distal part of the shaft and tip of the FIG. 11 dilator.

FIGS. 14 and 15 show cross sectional views of the handle assembly of the FIG. 11 dilator which provides force feedback information to the user.

FIGS. 16 and 17 schematically illustrate the visual force feedback information provided during use of the FIG. 11 dilator over a conventional endoscope.

FIG. 18 schematically illustrates the correspondence between the scale markings and the outer diameter of the dilatation surface of the FIG. 11 dilator.

FIGS. 19A and B are side views in partial section of the FIG. 14 safety handle mounted on the dilator body via a compression fitting.

FIGS. 20A and B are side sectional views of another embodiment of a safety handle for a dilator.

FIG. 21 is a side sectional view of yet another embodiment of a safety handle for a dilator.

FIG. 22 is a side sectional view of yet another embodiment of a safety handle for a dilator.

FIG. 23 is a side sectional view of yet another embodiment of a safety handle for a dilator.

FIG. 24 is a side sectional view of yet another embodiment of a safety handle for a dilator.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is hereby intended. Alterations and further modifications in the illustrated devices, and such further applications of the principles of the invention as illustrated herein are contemplated as would normally occur to one skilled in the art to which the invention relates.

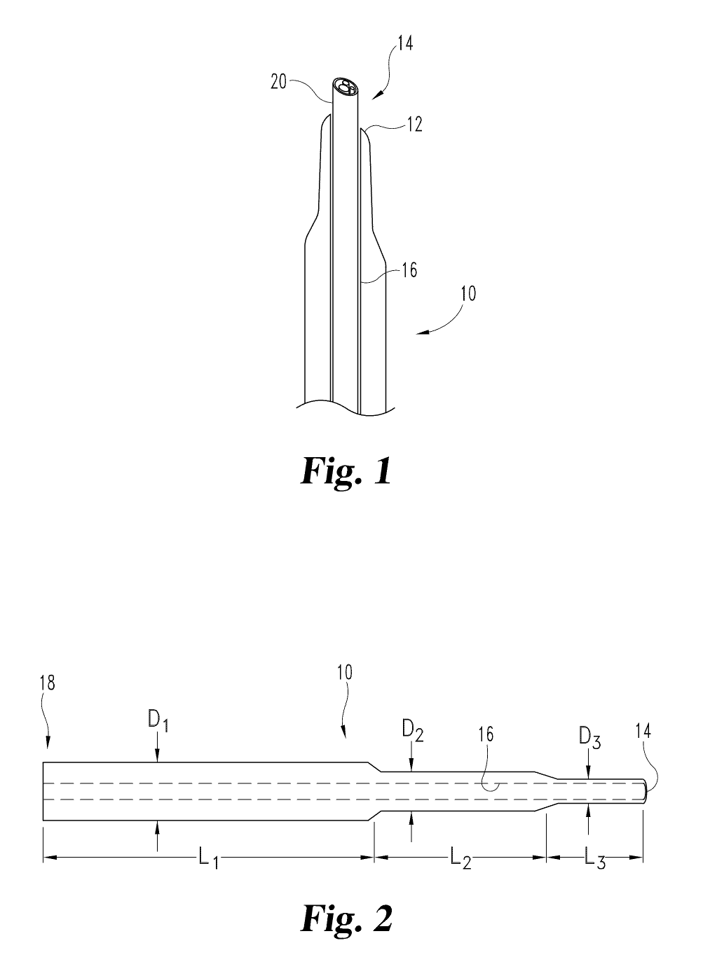

Referring to FIG. 1, a dilator 10 with an endoscope 20 extending through an endoscope lumen 16 is illustrated. Endoscope lumen 16 extends to opening 14 in the distal end 12 of dilator. The endoscope lumen 16 is sized and configured to allow passage of the endoscope 20 along the length of the lumen 16 and out of end 14. This configuration allows the endoscope 20 to serve as guide to the dilator 10 during insertion of the dilator into the patient and/or during dilation of a stricture.

In certain applications, the endoscope 20 will be a conventional 5 mm or 10 mm outer diameter endoscope and the dilator 10 will be constructed from a soft silastic material that provides a smooth inner surface for the endoscope lumen 16. Dilator 10 would typically be configured with an outer diameter that increases in size at increasing distances from the distal end 14. As shown in FIG. 2, the increase may be in stepwise fashion, and the relative dimensions of these sections may vary depending on the application. For example, with continued reference to FIG. 2, suitable dimensions for dilating esophageal strictures may be that the proximal section has a length L1 of about 45 cm and an outer diameter (D1) of 20 mm. The intermediate section may have a length L2 of 10 cm and a outer diameter (D2) of about 16 mm. The distal-most portion may be about 10 cm in length L3 and about 12 mm in outer diameter (D3).

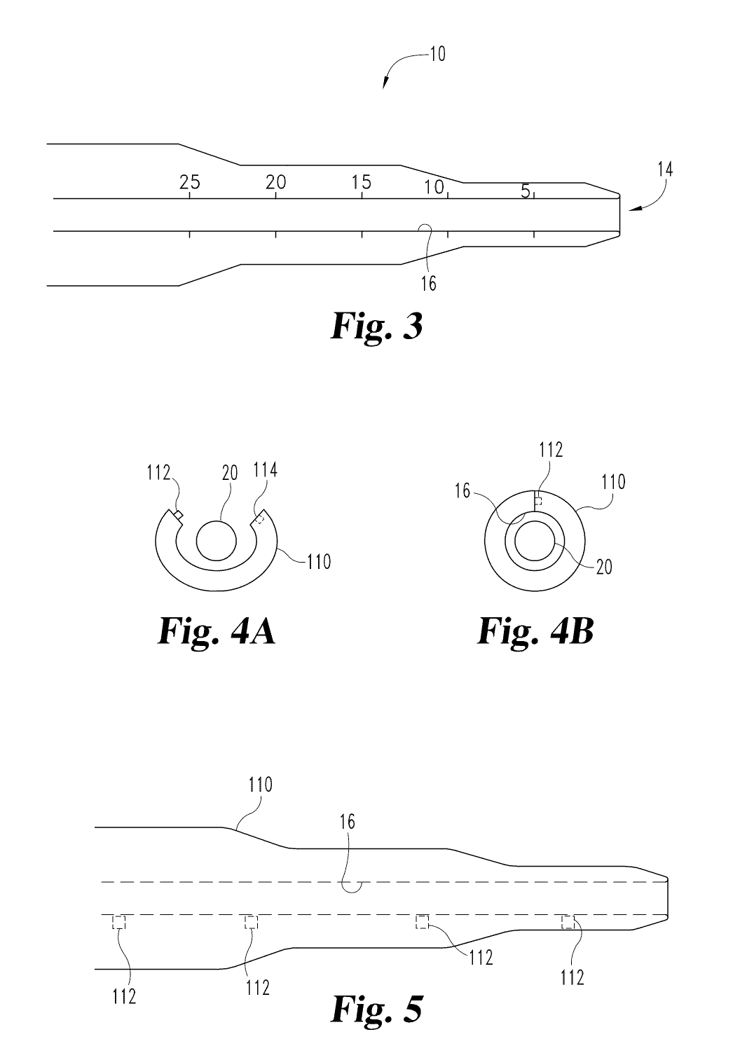

As shown in FIG. 3, gradations may be provided along the length of lumen 16. The gradations may be visible to the endoscope in the lumen and may serve to indicate the distance from the distal end 14. During use, these markings can be used to assist the operator in determining the relative location of the dilator in the patient. In addition to written numbers or letters to indicate position, the gradations or markings can be positioned and/or colored to visually indicate section breaks in the dilator. For example, the markings in the intermediate diameter portion can be rotated 120° from the markings in either the small or large diameter sections. This can assist the operator in rapidly making assessments of relative positioning of the components.

The dilator can be constructed such that is can be snapped, clamped, or otherwise attached to the endoscope anywhere along the length of the endoscope (i.e. not just being inserted over the distal end of the endoscope). As shown in FIGS. 4A, 4B, and 5, dilator 110 can be constructed with a longitudinal section that allows the lumen 16 of the dilator 110 to open into a C shape. When in the open C-shape configuration of FIG. 4A, the dilator is fit over the endoscope 20, and then locking tabs 112 are fit into locking recesses 114 to securely close the dilator 110 around the endoscope as shown in FIG. 4B. FIGS. 9 and 10 show alternative configurations for the orientation of the section line 50, 60 along which the dilator 10 is split to provide access to lumen 16. To reform lumen, the section lines 50 and 60 may be coated with an adhesive, hook and loop fasteners, or other attachment means that would facilitate securing the pieces together. Alternatively or in addition, a pull wire of string can be incorporated along the length of the dilator 10 to facilitate its easy removal from the endoscope in tear off fashion.

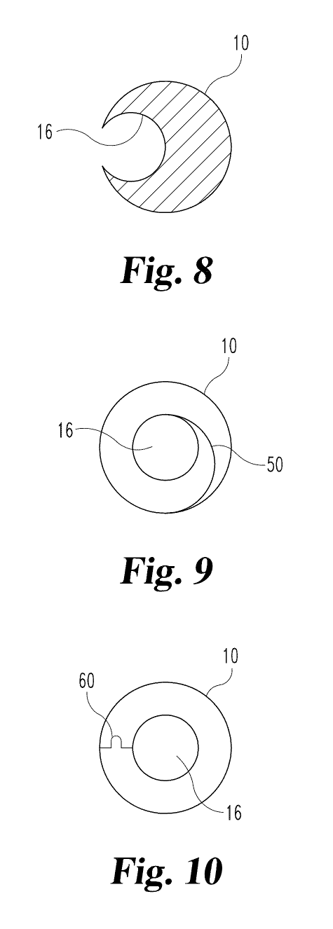

FIG. 8 shows a variation on this approach wherein the endoscope lumen 16 runs along the side of the dilator 10 and remains open along a length of the dilator 10. In this variation, the endoscope may be press fit into the lumen 16.

Instead of providing dilator 10 as a single integral unit, dilator 10 can be constructed from a number of different sections arranged in end to end fashion along the length of the endoscope. Each section could be attached over the endoscope independently. Adjacent sections could then be secured together during use, for example with adhesive. When constructing a dilator from individual sections, the sections can be chosen to have different outer diameters so as to construct a dilator tailored to the specifics of the current procedure. For example, it is envisioned that a kit would be sold having custom dilator sections including the following sizes 36, 39, 42, 45, 48, 51, 54, 57, and 60 F.

In addition to or in place of using the endoscope to guide insertion of the dilator into the body, the endoscope may act as a guide for the dilator during the actual dilation of a stricture or any other obstruction in a body lumen requiring dilation. For example, a preferred technique for dilating a stricture is to insert the endosope into the stricture and then to slide the dilator over the endoscope to dilate the stricture. This can be done while visualizing with the endoscope.

It is believed that cannulation of the stricture by the endoscope under direct visualization will reduce the chance that the dilator will become kinked at the obstruction site. Furthermore, because the endoscope has sufficient rigidity to prevent the mounted dilator from kinking, the stricture can be visualized in between applications of different gradations of the dilator to determine if there is excessive injury to the stricture area. Finally, because the endoscope and dilator can be moved around as a unit, additional strictures or obstruction areas can be treated with relative ease.

For example, in one contemplated procedure, the operating portion of the upper endoscope is 120 cm long and 100 to 110 cm is used to cannulate the pylorus before entering the duodenum. Duodenal examination is a routine portion of every endoscopy and is usually done first. If a stricture is anticipated by symptoms of difficulty swallowing (dysphagia) the dilator could be mounted on the endoscope and the dilation preformed to the desired level. With the dilator through the stricture the final portion of the examination can be completed. Thus only one cannulation for dilation would typically be required.

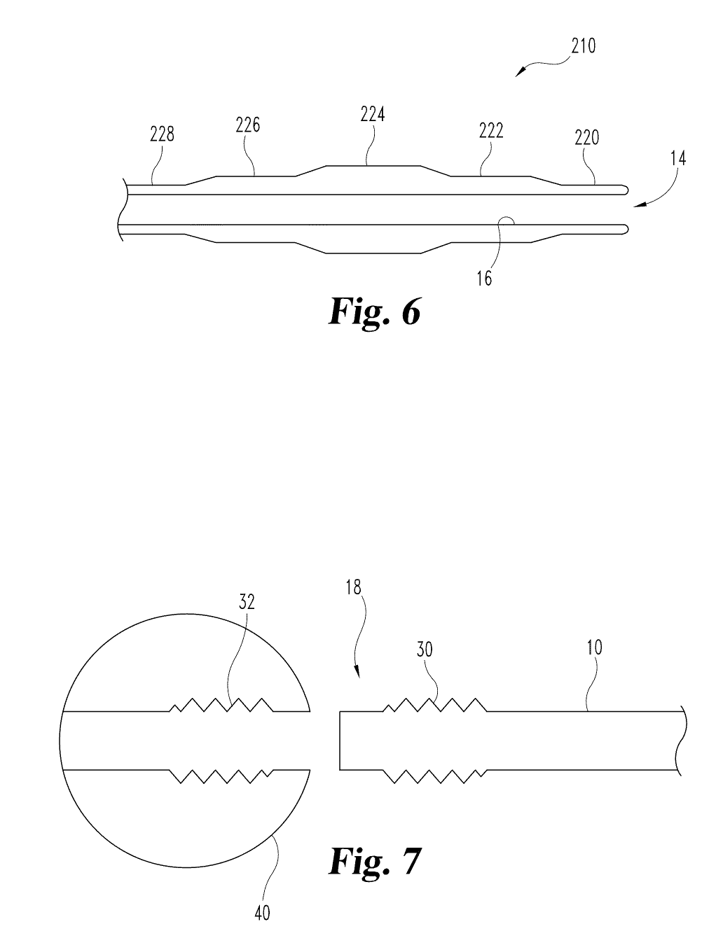

Referring to FIG. 6, a dilator with reverse gradation is illustrated. The outer diameter is greatest in dilation sections 224 and is gradually reduced both in a proximal direction (226, 228) and in a distal direction (222, 220). Inclusion of this reverse gradation can be useful to maintain a better airway. For example, a 60 F dilator in the oral pharynx for a prolonged time can cause low oxygen levels (hypoxia) that can be dangerous. Use of reverse gradations at the oral pharynx reduces the diameter of the dilator in the oral pharynx and should decrease the risk of hypoxia.

In another advantageous form, the present invention provides a dilator having a feedback mechanism for indicating when the applied force exceeds a predetermined limit. This can function as a safety check against the operator applying too much axial pressure to the dilator and causing a rupture. The feedback mechanism can take the form of a breakaway connection 32, 30 (FIG. 7) between a handle 40 and a distal section 18 of the dilator body 10 wherein the connection is configured to breakaway when the force applied between the handle and the dilator body exceeds a predetermined limit (e.g. 2 lbs). The breakaway level can be controlled by specifying the hardness or softness of one set of serrations 32, 30. Alternatively, a ball detent mechanism or lever spring can provide an engagement between the outer handle and the dilator which yields upon application of a predetermined force. Alternatively or in addition, the feedback mechanism can take the form of a sensor that measures the force and generates a signal when the force exceeds a predetermined limit.

It is to be appreciated that the principles described herein can be applied to any procedure where a constricted body lumen need dilation as well as in other devices and applications. For example, the dilator could serve as an overtube for suctioning blood during UGI bleeding. Such an overtube could be configured with a suction port fitting and an endoscopic channel specific for blood aspiration.

In another example, an O ring valve can be provided at the proximal end of the dilator to prevent the escape of insufflated air.

In another example, a device can be configured to function as a tamponade device during esophageal varieal bleed as the stomach is endoscoped. Such a device can then act as an overtube for multiple rubber band ligations of the varicies.

In another example, a device can be configured to function as a dilator for malignancies and then as an overtube for endoscopic ultrasound fine needle aspirations, which may otherwise require multiple intubations.

In another example, the device can function as a port for colonoscopy and multiple polyp excision.

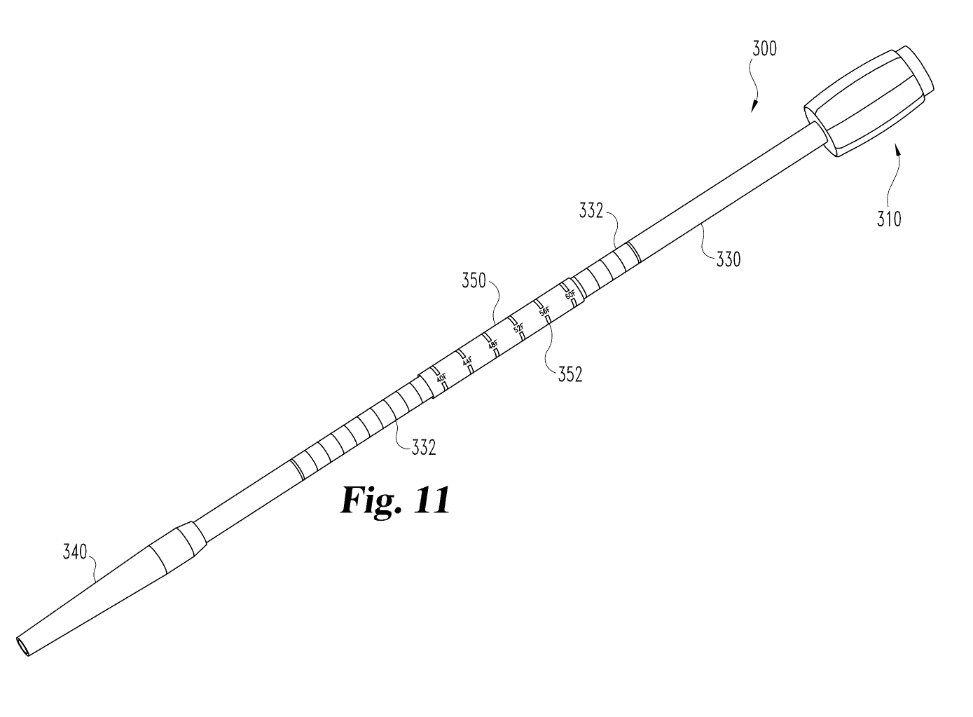

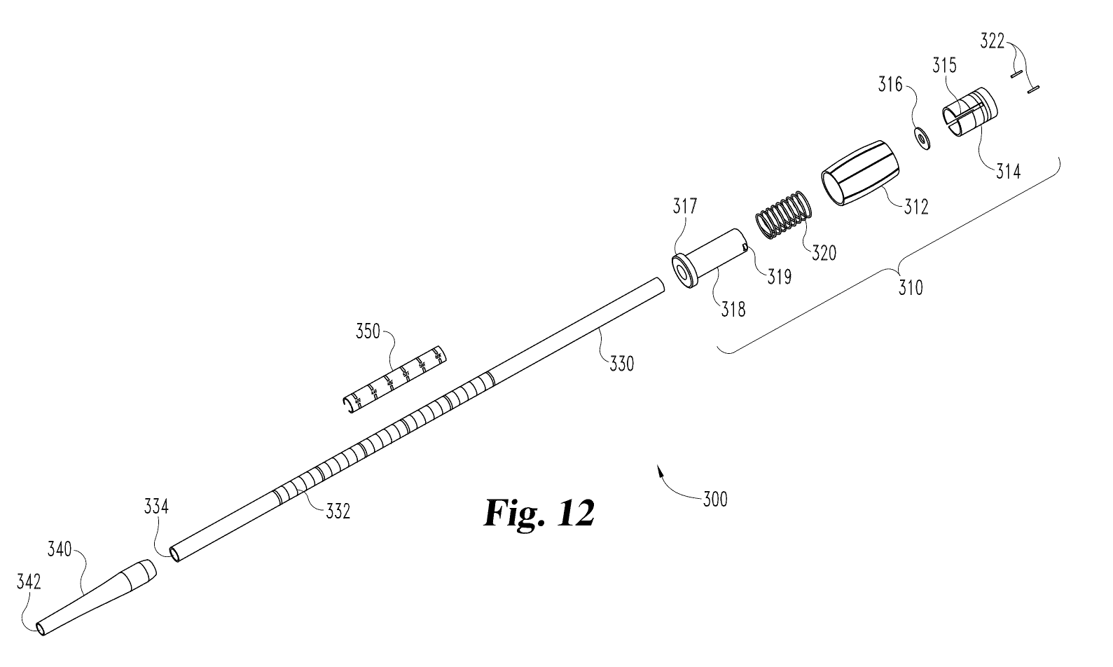

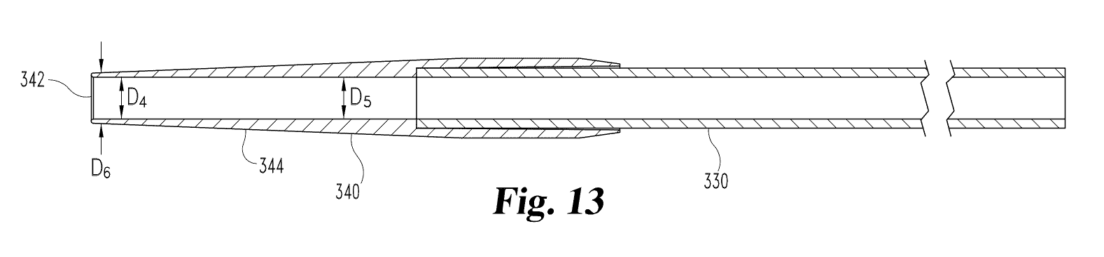

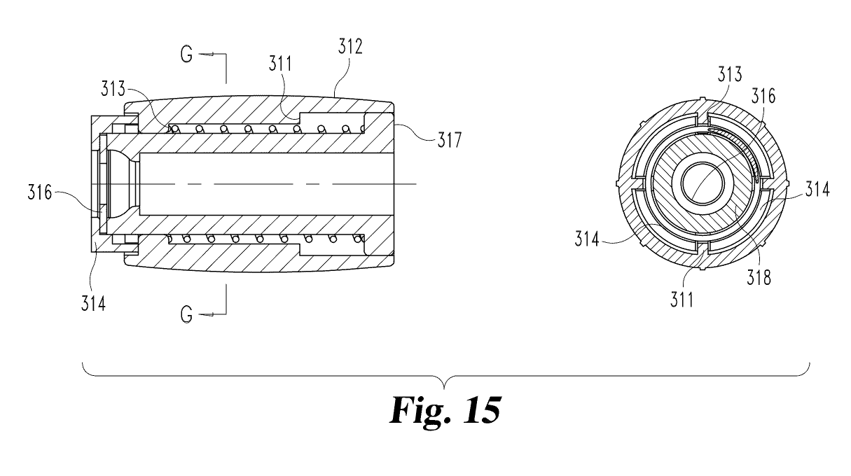

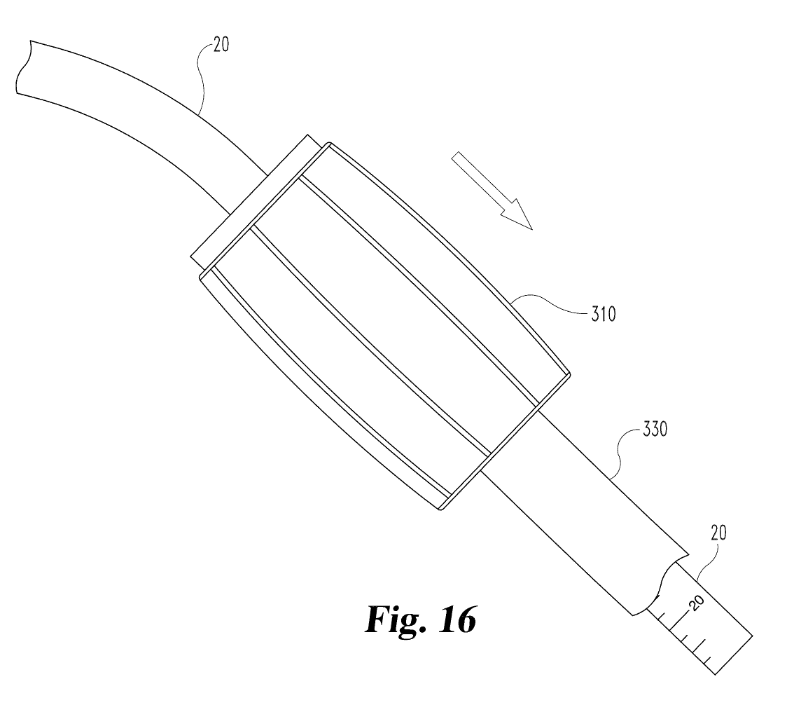

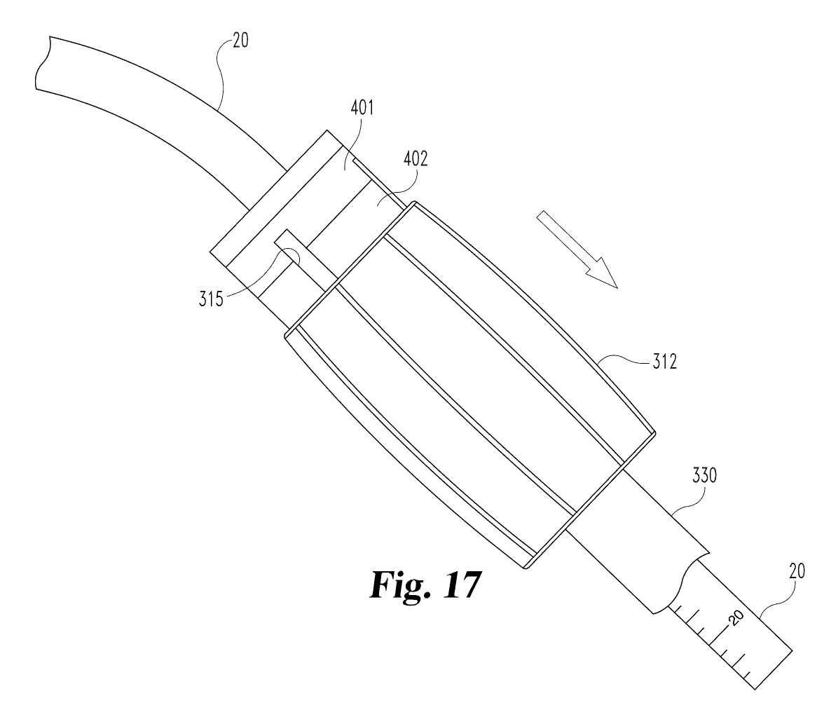

Referring now to FIGS. 11-18, another embodiment of a dilator according to the present invention is depicted. Dilator 300 includes an elongated body 330 having a handle 310 mounted at its proximal end and a dilating tip 340 mounted at its distal end. An endoscope lumen extends the entire length of dilator 300 such that it may be used in over the endoscope operations as described above.

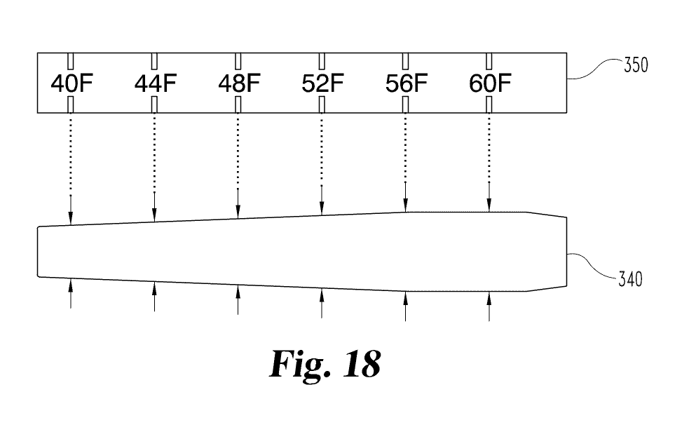

The dilating tip 340 includes a dilating surface 344 surrounding the distal exit 342 of the endoscope lumen. As illustrated, surface 344 is continuously tapered towards the distal end. A dilation scale 350 is slidably positionable along the length of the body 350. The scale 350 has markings 352 that correspond to the outer diameters of the dilating tip 340, as shown in FIG. 18. In other words, the axial distance between the 40 F and 60 F markings on scale 350 corresponds to the axial distance between the 40 F and 60 F portions of the dilating surface 344. This correspondence allows the scale to be used, as explained in more detail below, to measure how much a particular stricture is being dilated. The dilator body 330 also includes a series of distance markings 332 indicating distance to the distal tip of the dilator, which may be used during initial positioning of the scale 350.

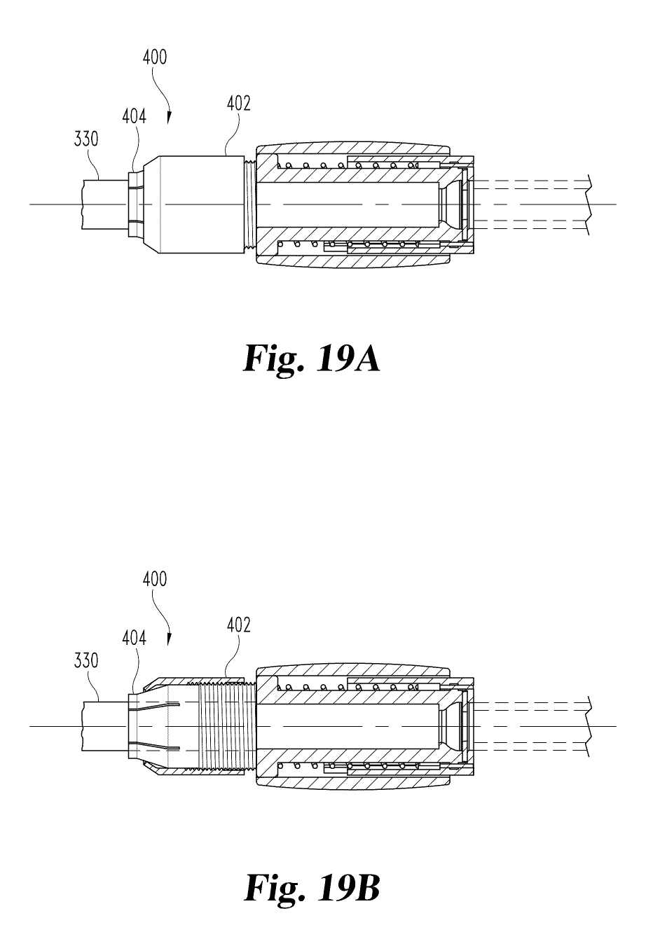

The handle 310 is designed to give tactile and visual feedback to the operator with respect to applied axial force. The handle 310 includes an outer handle 312, an inner handle 318 and a handle cap 314. The inner handle 318 is rigidly secured to the proximal end of the elongated body 330, for example with glue or via a compression type fitting (see FIG. 19). A compression spring 320 fits over the inner handle 318 and rests at one end against flange 317. The other end of spring 320 is in contact with ribs 313 that project inwardly from the interior of the outer handle 312. These ribs 313 are received in corresponding longitudinally extending slots 315 in the handle cap 314.

During assembly, the handle cap 314 is placed over the inner handle 318 and rotated to seat ears 319 in a supporting channel or slots (not shown) formed in the interior of cap 314. Pins 322 are placed through cap 314 and ears 319 to retain them together and thereby rigidly couple cap 314 to the handle 314. A resilient annular ring 316 is contained between the inner handle 318 and the handle cap 314 and, in use, forms a seal about the portion of the endoscope 20 that extends distally from the dilator. The inner diameter of the annular ring 316 is smaller than the inner diameter of the endoscope lumen such that the ring 316 may contact and form a seal against the endoscope.

It is to be appreciated that, because the cap 314 is rigidly coupled to the inner handle 318, which is in turn rigidly coupled to the shaft 330, torsional force applied to the outer handle 312 will be transmitted to the shaft 330 via the inwardly projecting ribs 313 in the longitudinal slots 315. Axial force applied to the outer handle 312 (in the direction of the arrow in FIGS. 16-17) will be transmitted to the shaft 330 via spring 320, which will compress and cause force markings 401, 402 (FIG. 17) to be revealed in correspondence to the degree that the shaft 330 experiences resistance to axial motion. Accordingly, as axial force is being applied to outer handle 312 during dilation of a stricture, the outer handle 312 will begin to depress spring 320 and therefore slide longitudinally to reveal markings 401, 402. Markings may be color coded or otherwise preconfigured to indicate how much force is being applied. Revealed color stripes are a form of visual feedback to the operator of the amount of applied force.

It is to be appreciated that the handle 312 can also depress the spring 320 to such an extent that portion 311 of the ribs that project inwardly from outer handle 312 contact flange 317 in a positive stop. This positive stop provides tactile feedback to the operator about the applied force. In preferred implementations, the handle is designed to provide warnings so that the operator does not place more than about 2 lbs of axial force, for example with the positive stop activated at 2.5 lbs of applied force. The handle may also be designed to provide audible feedback, for example by incorporating appropriate sensors in the inner and outer handles.

In use, the operator mounts the dilator 300 on the proximal aspect of an endoscope 20, preferably after thoroughly lubricating the outer surface of the endoscope with a suitable lubricant, such as a clear water based lubricant. With the patient sedated, the endoscope 20 (with the dilator assembly mounted proximally) is introduced into the esophagus via the mouth or nose. At this stage, the operator may want to use the endoscope in any conventional fashion, for example to examine the esophagus and stomach and perform biopsies.

When the operator identifies a lesion or stricture that requires dilation, he uses the distance markings that are conventionally provided along the length of the endoscope 20 (see FIG. 16) to determine the stricture location. To determine location, the operator positions the distal end of the endoscope at the site of interest and reads the length indicated on the endoscope body at an external reference point. Any fixed reference point may be used. For a transoral procedure, the patient would be provided with a bite block, and the proximal edge of the bite block (not shown) would serve as a convenient external reference point. For a transnasal procedure, the entrance to the patient's nostril is a convenient reference point.

Having determined from the endoscope how far (length) into the patient the stricture is located, the operator places the distal aspect of the dilation scale 350 at the corresponding location on the dilator 333 shaft, as indicated by the lengths markings 332. In other words, if at endoscopy, the obstructing lesion is determined to be 38 cm from the bite block, the operator places the distal end of the dilation scale 350 at the 38 cm marking on the dilator shaft 330. The dilation scale 350 is clamped or otherwise affixed in place on the dilator body such that it remains in this position on the dilator shaft 330 for the remainder of the procedure.

Having ascertained the stricture location and transferred that measurement to the dilator, the operator passes the endoscope through the stricture. When treating an esophageal stricture, the operator may choose to straighten the endoscope into the stomach.

With the endoscope now functioning as a guide for the dilator, the operator advances the dilator 300 through the upper esophageal sphincter (the proximal esophagus) and into the esophagus to the point of the stricture. Since the dilation scale 350 has been placed to mark the beginning of the stricture, the operator will know he has reached the stricture when the distal aspect of the scale 350 reaches the predetermined external reference point (e.g. bite block).

Having determined that he has reached the stricture, the operator proceeds to dilate the stricture. For safety, the operator uses the handle 310 to apply the axial force to the dilator during dilation of the stricture. By grasping the outer handle 312, the operator is able to apply axial force to advance the dilator through the stricture while receiving feedback as to the amount of applied force. As illustrated in FIGS. 16 and 17, as axial force is applied, the spring 320 depresses to selectively reveal a plurality of force indicators 401 and 402, which may include a numeric scale as well as or in place of colored bands. The first indicator 401 to be revealed may be correlated with a safe amount of applied force (e.g. under 2 lbs of applied force) with the second indicator indicating an unsafe amount of force (e.g. greater than 2 lbs of applied axial force). The operator monitors the visual indicators in an effort to avoid over-exertion, and the operator can tell when an unsafe level of force is being applied so as to reduce the force or stop dilating altogether, thereby reducing the chances of rupture.

It is to be appreciated that the dilator handle may be used to rotate the dilator shaft 330 if the occasion arises and/or the operator may grasp the shaft 330 directly to facilitate rotation. Rotation of the shaft 330 is useful to assist manipulation of the dilator along a tortuous path. The dilating surface may also be non-uniform radially (e.g. oval in cross section), in which case rotation of the shaft is important to be able to reposition the dilating surface into the desired configuration.

While the dilator may not have passed entirely through the stricture, it will typically have been advanced some distance and therefore accomplished some amount of dilation. The amount of dilation can be determined by scale 350, which functions similar to a depth indicator but which has marking that, as illustrated in FIG. 18, correspond to outer diameters of the dilator. In other words, if at the point dilation stops, the 48 F marking has been advanced to where the 40 F marking was initially (i.e. the proximal face of the bite block), then the operator knows that the stricture was dilated to 48 F.

Upon completion, the operator pulls the dilator back on the proximal shaft of the endoscope and then pulls the endoscope out slowly, inspecting the area of dilation for any complications. The entire assembly is then removed and the procedure is complete.

It is to be understood that the shaft and tip of the dilator may be constructed of conventional materials plastic material, such as the materials used in the commercially available Savory Gillard dilator, and may be assembled from parts of or configured as a unitary whole. Another suitable material for construction of the shaft and distal tip may be obtained from Alpha Gary, such as their 2235 L/FS 85S Blue 7652 (Alpha Gary Item # 044168).

The inner diameter of the dilator should be selected to closely approximate the outer diameter of the endoscope. In general, it is desirable to have sufficient clearance for the dilator to slide on the endoscope without a resistance of its own, but too large of a gap between the endoscope and the dilator at the distal end 342 could cause the lining of the esophagus to become trapped and create injury by stripping the mucosa. Applicants have found that, to accommodate many commercially available 10 mm endoscopes, the inner diameter D4 of the dilator at the distal tip may be approximately 10.1 to 10.5 mm, for example between 10.1 and 10.2, between 10.2 and 10.3 mm, between 10.3 and 10.4, between 10.4 and 10.5 mm, or between 10.3 and 10.5 mm at or near the distal end 342. In other variations or where other endoscope sizes are used, it may be desirable to have the inner diameter of the dilator within about 0.5 and 3 mm of the outer diameter of the endoscope, for example within 1.5 to 2.5 mm. For example, dilators could be constructed for use with pediatric (9 mm) or transnasal (7 mm) endoscopes.

For clearance, the inner diameter of the endoscope channel may be larger proximally, for example by configuring the tip with a slight taper (i.e. D5 greater than or equal to D4). The distal edges of tip forming opening 342 are preferably full rounds, with a radius less than about 1 mm, for example having a radius of 0.7 mm. Accordingly, it is to be appreciated that in certain embodiments, the outer diameter D6 of the distal tip of the dilator will be only slightly larger than the inner diameter D4, which in turn will be only slightly larger than the outer diameter of the endoscope. For example, the difference between D6 and D4 may be less than 6 mm, 5 mm, 4.5 mm, 4 mm, 3 mm, or 2.5 mm.

The dilator may be sized and configured such that it can be mounted on the proximal aspect of the endoscope and kept out of the way (e.g. out side the patient) until needed. For example, where, as in the illustrated embodiment, the endoscope lumen extends the entire length of the dilator, it may be useful for the overall length of the dilator to be less than about 70 cm, for example in the range of 55-65 cm. To allow the markings on the endoscope to be read even if the dilator is covering them, sections of the dilator may optionally constructed of transparent materials.

Dilator 300 may be used for a variety of dilation applications. It is expected that it will be particularly effective for dilating esophageal lesions related to cancer, a GERD caused peptic stricture, a radiation stricture, a caustic stricture, an inflammatory stricture caused by an allergy (eosinophilic esophagitis), a spastic upper or lower esophageal sphincter, or an esophageal web.

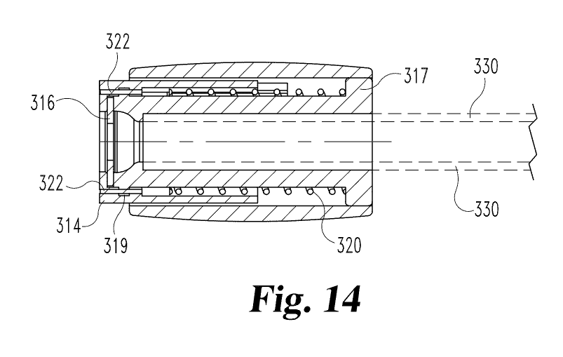

It is to be appreciated that the force feedback handle can be provided integrally with a given dilator for one time use applications. Alternatively, the handle may be provided independently from any particular dilator. FIGS. 19A and 19B illustrate the safety handle of FIG. 14 adapted so as to be mountable on a conventional esophageal dilator. As illustrated, the inner handle is provided with a compression fitting 400 that is inserted over the end of an esophageal dilator body, which is typically a 40-60 French outer diameter tube, and secured via captive nut 402. Tightening the captive nut 402 compresses flanges 404 against the outer surface of the dilator body 330 so as to secure the inner handle to the dilator. The handle may be removably attached to the dilator body in other ways, such as via set screws or some other type of fitting suitable for establishing a secure connection between the handle (e.g. the inner handle 318) and the dilator body.

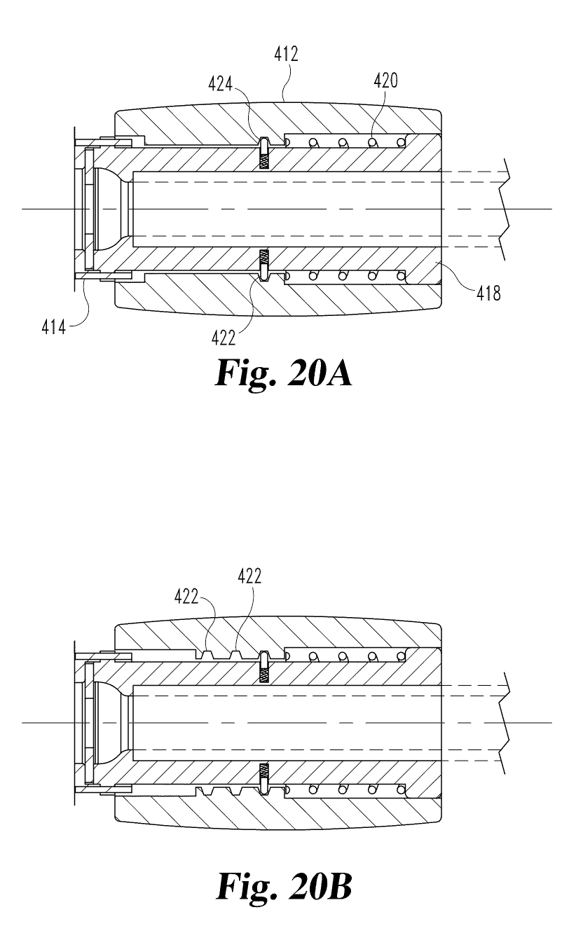

It is also to be appreciated that the safety handle can be designed so as to provide force feed back in a variety of ways or combinations of ways, including tactile feedback, audible feedback, and/or visual feedback. For example, FIGS. 20A and 20B illustrate embodiments of a safety handle in which the inner and outer handles are yieldingly engaged via a plunger and indent arrangement. At low applied axial forces, the plunger 424 mounted in the inner handle 418 is engaged in the indent 422 in the outer handle 412, and therefore the inner and outer handles 418, 412 are relatively fixed. However, when the applied axial force is sufficient to dislodge the plunger 424 from the corresponding indent 422, the outer handle 412 will translate axially relative to the inner handle 418, compressing the dampening spring 420 and providing a form of tactile feedback. The axial translation of the inner and outer handles also causes markings 414 on the inner handle to be revealed, providing a form of visual feedback.

FIG. 20B illustrates an embodiment wherein a series of indents are provided in the outer handle so as to provide a number of different engagements positions for the inner and outer handles. Since each different engagement position corresponds to a different amount of compression of the spring 420, the amount of axial force needed to dislodge the plunger 424 from successive indents 422 can be made to increase. The angle and/or depth of the indents 422 may also be adjusted to provide a desired force-response profile. The engagement of a plunger 424 into a corresponding recess 422 may be designed so as to produce an audible click, providing a still further form of feedback. Alternatively, only a single set of indents are provided, as depicted in FIG. 20A. It is to be understood that the spring 420 may also function as a return spring by forcing the inner and outer handles into their original positions once the axial force is released.

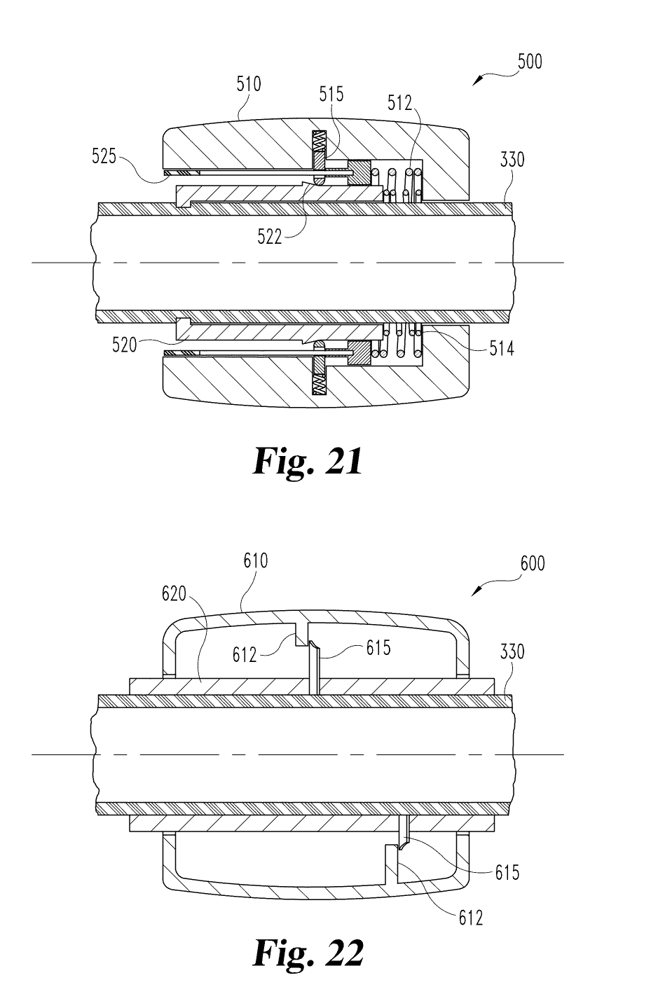

FIGS. 21 and 24 illustrate embodiments of a safety handle which are designed to trigger a warning flag at the handle to provide visual feedback of excessive axial force. The spring loaded warning flags are initially hidden beneath the outer handle and project outward when triggered. The triggers are mechanical triggers which are activated by appropriate cams when a sufficient force is applied to the outer handle to compress a small displacement spring. Electronic triggers based on force transducers or electrical contacts may also be employed, either to provide feedback at the handle (such as by producing vibration or activating a light or sound) and/or remotely (such as by transmitting a signal for display on a monitor).

In FIG. 21 the inner handle 522 is secured to the dilator body 330 and a warning flag 525 is spring loaded between the inner 522 and outer 510 handles by spring 512. The flag 525 is attached to a stepped pin which is maintained in its undisplaced position (shown in FIG. 21) by plunger 515. A small displacement spring 514 is compressed when excessive axial force is applied to the outer handle, causing the plunger 515 to be displaced upwardly by ramp 522 and triggering the pin/warning flag 525 to be ejected by spring 512.

In FIG. 24, the firing spring 752 for warning flag 742 is provided as a part of the inner handle 760. More specifically, when the small displacement spring 750 is compressed by excessive axial force, cam surface 742 on outer handle 740 releases the lever from one of the grooves and allows spring 752 to trigger flag 742. As illustrated, there are a plurality of different grooves and different colors on the pin, which may be used to provide a multi-stage firing mechanism.

FIG. 22 illustrates the use of springs 615 to provide yielding engagement between the inner 620 and outer 610 handles. Springs 615 are leaf springs which extend radially from the inner handle 620 (which is secured to the dilator body 330) to engage corresponding interface flanges 612 in the outer handle 612. The leaf springs 615 are curved such that they resist bending until a critical force is applied, at which point they bend and release their resistance to axial translation (and optionally produce a sound). Springs with different characteristics can be spaced at different radial positions so as to engage different interface flanges to further tailor the force response characteristics.

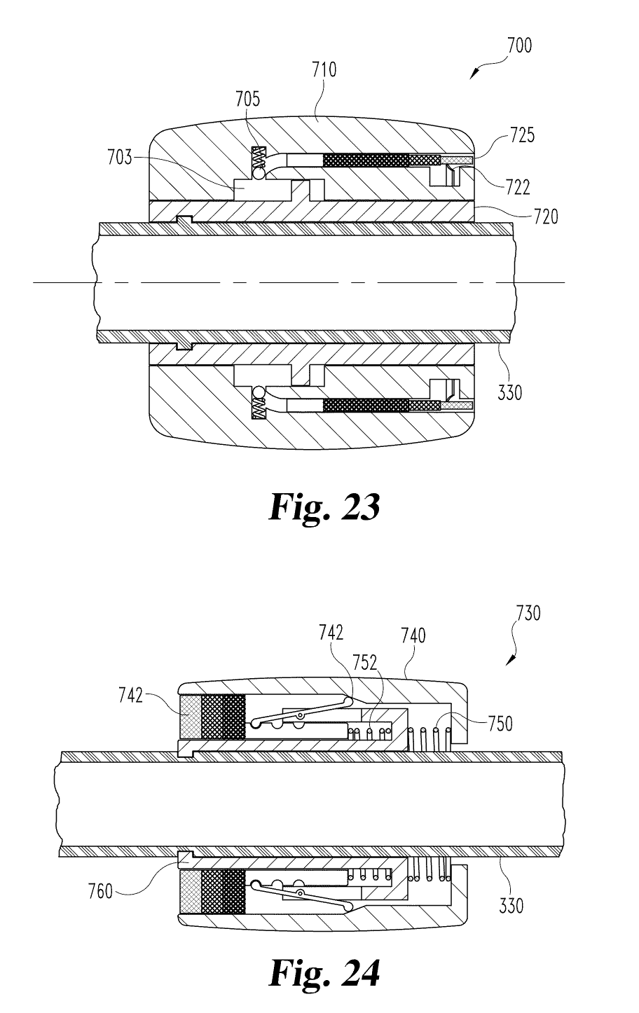

FIG. 23 illustrates the use of a pneumatic valve to trigger a visual and/or audible warning based on the applied axial force exceeding a threshold. Fluid is provided in a cavity 703 between inner 720 and outer 710 handles. The cavity is compressed by relative axial translation of the inner and outer handles, which displaces ball 705 and exerts pressure on flag 725, overcoming the retaining force of leaf spring 722.

In still further variations, the relative movement between the inner and outer handles can be utilized to puncture a seal to release and odor or activate a color change. As noted previously, the safety handle may also be provided with a force sensor, such as a piezoelectric element, to electrically sense the applied axial force and provide an electrical signal to warn of over exertion.

While the invention has been illustrated and described in detail in the drawings and foregoing description, the same is to be considered as illustrative and not restrictive in character. Only certain embodiments have been shown and described, and all changes, equivalents, and modifications that come within the spirit of the invention described herein are desired to be protected. Any theory, mechanism of operation, proof, or finding stated herein is meant to further enhance understanding of the present invention and is not intended to limit the present invention in any way to such theory, mechanism of operation, proof, or finding. Thus, the specifics of this description and the attached drawings should not be interpreted to limit the scope of this invention to the specifics thereof. Finally, all publications, patents, and patent applications cited in this specification are herein incorporated by reference to the extent not inconsistent with the present disclosure as if each were specifically and individually indicated to be incorporated by reference and set forth in its entirety herein.

What is claimed is:

1. A dilator comprising:
a dilator body having a distal end and at least one endoscope lumen open to the distal end, wherein the endoscope lumen is sized to allow an endoscope positioned in the lumen to extend out the distal end of the dilator, wherein the distal end of the dilator defines an outer diameter and an inner diameter having a difference therebetween of less than about 4 mm.

2. The dilator of claim 1 wherein the dilator body defines an exterior dilating surface having a non-uniform outer dimension.

3. The dilator of claim 2 wherein the exterior dilating surface has at least three areas of different outer dimension.

4. The dilator of claim 3 wherein the areas increase in outer dimension as they increase in distance from the distal end of the dilator.

5. The dilator of claim 3 wherein the areas decrease in outer dimension as they increase in distance from the distal end of the dilator.

6. The dilator of claim 2 wherein the dilating outer dimension initially increases at increased distances from the distal end of the dilator and then decreases.

7. The dilator of claim 6 wherein two different areas of successively smaller outer dilating dimension are proximal to an area of maximum dilating dimension.

8. The dilator of claim 1 further comprising:
a handle for applying axial force to the dilator; and
a feedback mechanism for indicating when the applied force exceeds a predetermined limit.

9. The dilator of claim 8 wherein the feedback mechanism includes a yielding engagement between the handle and the dilator, the engagement configured to yield to an applied force that exceeds the predetermined limit.

10. The dilator of claim 8 wherein the feedback mechanism includes a force sensor that generates a signal when the force exceeds the limit.

11. The dilator of claim 1 wherein the dilator body includes a plurality of body segments attached together over an endoscope.

12. The dilator of 8 wherein the feedback mechanism includes a biased grip that slides to reveal visual indicators of applied force.

13. The dilator of claim 12 wherein the feedback mechanism includes a positive stop.

14. The dilator of claim 8 further comprising a scale slideably positionable along the length of the dilator.

15. The dilator of claim 14 wherein the scale includes visual indicators of dilation diameter spaced along its length in correspondence to dilation diameters provided by the distal end of the dilator.

16. The dilator of claim 15 wherein the distal end of the dilator is gradually ramped.

17. A method for delivering a dilator to a stricture comprising:
providing an endoscope at the site of the stricture; and
sliding the dilator over the endoscope to the site of the stricture.

18. The method of claim 17 further comprising:
determining a relative distance to the stricture with the endoscope; and
positioning a scale along the length of the dilator based on the determined distance to the stricture.

19. The method of claim 18 further comprising:
dilating the stricture; and
determining the amount of dilation based on the scale.

20. The method of claim 17 wherein dilating the structure includes applying axial force to the dilator via a handle that supplies feedback based on the amount of applied axial force.

21. The method of claim 20 wherein the feedback is tactile feedback.

22. The method of claim 20 wherein the feedback is visual.

23. The method of claim 22 wherein the handle slides to reveal visual indicators of applied axial force.

24. The method of claim 17 further comprising:
inserting the endoscope to the stricture while the dilator is mounted on the endoscope;
reading a marking on the endoscope while the dilator is mounted on the endoscope to determine a relative distance to the stricture; and
positioning a scale on the dilator based on the determined relative distance to the structure.

25. The method of claim 24 wherein the marking on the endoscope is read through a portion of the dilator.

26. The method of claim 24 wherein the distal tip of the dilator is located distal to the marking that is read.

27. The method of claim 24 further comprising:
inserting the endoscope through the stricture; and
dilating the stricture by sliding the distal end of the dilator over a portion of the endoscope that is within the stricture.

28. A method for dilating a stricture comprising:
inserting an endoscope into the stricture; and
advancing a dilator over the endoscope to dilate the stricture.

29. The method of claim 28 wherein the dilator is advanced by applying axial force to a handle of the dilator that gives feedback when the applied axial force exceeds a predetermined limit.

30. A dilation system comprising:
an endoscope having an elongated endoscope body having distance markings thereon and a distal end; and
a dilator having an elongated dilator body having a distal end;
wherein the endoscope body is coaxially within the dilator body and the distal end of the dilator is proximal to the distal end of the endoscope.

31. The dilation system of claim 30 wherein the dilator further comprises a handle for delivering axial force to the dilator, the handle configured to provide feedback to the user when the applied axial force exceeds a predetermined amount.

32. The dilation system of claim 31 wherein the handle includes an outer handle body that slides relative to an inner handle body based on the amount of applied axial force.

33. The dilation system of claim 30 wherein there are distance markings on the dilator body, the system further comprising a scale slidably positionable along the length of the dilator body, the scale having markings corresponding to the dilation diameters of the dilator.

34. The dilation system of claim 30 wherein the dilator defines an endoscope lumen that extends along its entire length.

35. A safety dilator comprising:
an elongated body adapted to be inserted into a body orifice and used to enlarge a constricted body lumen by application of axial force to advance the body through the constricted lumen; and
a handle section adapted to transmit the axial force to the elongated body, wherein the handle section provides visual indications of the amount of applied axial force.

36. The safety dilator of claim 35 wherein the handle section includes an outer handle body that slides relative to an inner handle body based on the amount of applied axial force.

37. The safety dilator of claim 36 wherein the outer handle body is operable to transmit rotational force to the elongated body.

38. The safety dilator of claim 37 wherein the outer handle includes a plurality of inwardly projecting ribs positioned in sliding engagement in longitudinally extending slots in the inner handle body.

39. A handle for use with a dilator, wherein the dilator is adapted to be inserted into a body orifice and used to enlarge a constricted body lumen, wherein the handle is designed to transmit axial and rotational force to the dilator and wherein the handle provides feedback to the user when the applied axial force exceeds a predetermined amount.

40. The handle of claim 39 wherein the feedback is selected from tactile feedback, visual feedback, audio feedback, and combinations thereof.

41. The handle of claim 40 wherein tactile feedback is provided via relative translation of the handle and the dilator.

42. The handle of claim 39 wherein the handle is yieldingly engaged with the dilator.

43. The handle of claim 42 wherein the handle is yieldingly engaged with the dilator at a plurality of different force thresholds.

44. The handle of claim 39 wherein visual indications of applied axial force are provided.

45. The handle of claim 44 wherein the visual indications are triggered at specified force thresholds.

46. The handle of claim 45 wherein the visual indications are triggered mechanically or electrically.

47. The handle of claim 44 wherein the visual indications are provided at the handle.

48. The handle of claim 44 wherein the visual indications are provided on an electronic display remote from the handle.

49. A handle for use with a dilator, wherein the dilator is adapted to be inserted into a body orifice and used to enlarge a constricted body lumen by application of an axial force transmitted through the handle, wherein the handle is configured to translate axially relative to the dilator when the applied axial force is above a predetermined threshold.

50. The handle of claim 49 wherein the axial displacement of the handle and the dilators once the threshold is exceeded is based on the amount of applied axial force.

51. The handle of claim 49 wherein the axial displacement of the handle produces an audible noise so as to warn the operator that the force threshold has been exceeded.
